(19) United States
(12) Patent Application Publication
(10) Pub. No.: US 2012/0263852 A1
(43) Pub. Date: Oct. 18, 2012

(54) WHEY PROTEIN CONCENTRATE, ITS PREPARATION AND ITS USE

(75) Inventors: Suzanne Godelieve Thiessen-Bolder, Malden (NL); Linqiu Cao, Wagening (NL); Iliana Hidalgo Torres, Wageningen (NL)

(73) Assignee: Friesland Campina Nederland Holding B.V.

(21) Appl. No.: 13/501,207

(22) PCT Filed: Oct. 11, 2010
(86) PCT No.: PCT/NL2010/050670

§ 371 Date: Jun. 29, 2012

(30) Foreign Application Priority Data
Oct. 12, 2009 (EP) 09172797.4

(51) Int. Cl. C07K 1/34; C07K 14/47; G01N 21/75; A23L 1/317; A23L 1/325; A23L 1/31; A23G 3/44; A21D 13/00; C07K 1/36; A23C 21/00

(52) U.S. Cl. 426/549; 530/414; 530/350; 436/501; 426/583; 426/643; 426/641; 426/660; 426/646

(57) ABSTRACT

The invention pertains to process for manufacturing whey protein concentrate (WPC) from whey, said process involving (a) providing acidified whey; (b) increasing the pH of said acidified whey using one or more carbonate salt(s), preceded and/or followed by ultrafiltration, and (c) subjecting the ultrafiltered carbonate-containing whey to spray drying. A WPC is provided having improved functional properties, particularly increased gel strength and reduced salt sensitivity (i.e. meaning that the functional properties of the WPC are affected by salt to a lesser extent).

### FIELD OF THE INVENTION

The invention pertains to a dried whey protein concentrate (WPC) having improved functional properties, to a process for the manufacture of such WPC and to the use of such WPC in the manufacture of various foodstuffs, for instance as an egg white replacer in fish, meat and bakery applications.

### BACKGROUND DESCRIPTION

WO93/20713—its contents herein incorporated by reference—discloses a process for the manufacture of a whey protein concentrate from acid or sweet whey, which process comprises the steps of reducing the pH of the whey to a pH in the range of 2.5-3.5, followed by ultrafiltration, and optionally diafiltration for the further removal of lactose. Following ultrafiltration or diafiltration the pH of the retentate is raised to a pH in the range of 6.0-7.0, followed by spray drying. The pH adjustment is carried out using sodium hydroxide, potassium hydroxide or calcium hydroxide before spray-drying. Alternatively, the pH adjustment may be carried out before ultrafiltration, in which case the whey product still retains the desired gelling characteristics, but has the additional advantage of a reduced mineral content, due to subsequent partial removal of the added alkali mineral during ultrafiltration/diafiltration. The process is reported to result in the production of WPCs having protein content of the order of 80-90% by weight, with consistently improved functional properties. Whey protein concentrates are marketed as WPC80 according to this or similar concept. U.S. Pat. No. 4,362,761 teaches similarly.

GB 1,313,085 and EP 22.696 disclose processes for obtaining a protein concentrate from whey with the use of ultrafiltration. The whey is adjusted to a pH below the isoelectric point, thereafter subjected to ultrafiltration, heated to ensure microbial destruction before and/or after ultrafiltration, the product obtained then, if desired, being neutralised and optionally dried. GB 1,313,085 a concentrate at a dry mass content of about 30% was spray-dried. In EP 76 685 no neutralisation step is performed.

However, in the art a need continues to exist to further improve the functionality of WPCs, in particular in terms of gel strength and related properties. Although WO93/20713 claims to provide a process involving pH adjustment to pH 6.0-7.5, the preferred and disclosed embodiments are at conditions below pH 7. The cause rests in the fact that pH levels at 7 or higher result in premature gelling of the whey protein during the concentrating step and thermal processes involved in (spray) drying, or at least to an increased viscosity. Obviously, this premature gelling may lead to undesired blocking of the production lines before the final spray drying step. The effect of gelation at pH 7 is addressed in Boye et al. “Factors affecting molecular characteristics of whey protein gelation” Int. Dairy Journal 5 (1995) 337-353. The prior art does not provide measures to circumvent this premature gelling behaviour in manufacture. Outside the field of WPC preparation, XP002558495 teaches the use of UF-WPC which after its preparation has been adjusted to pH 9.5 using carbonates as a substitute for egg white in pie topping meringue. pH adjustments after preparation have little in common with the gelling issues during manufacture.

### SUMMARY OF THE INVENTION

It is an objective of the present invention to provide WPC having improved functional properties, particularly increased gel strength and reduced salt sensitivity (i.e. meaning that the functional properties of the WPC are affected by salt to a lesser extent). It is also an objective to provide an industrially applicable manufacturing process for preparing such WPC having improved functional properties, the process not being hampered by any premature gelling.

The inventors have found that the above mentioned goals can be achieved by modifying conventional ultrafiltration (UF)-based manufacturing methods, such as disclosed in WO93/20713, by performing the above mentioned pH adjustment prior to spray drying with carbonate salts. In terms of ash content the product remains practically unchanged compared to its alkali-based counterpart, but it is found that at least part of the carbonates in subsequent spray drying evaporates as CO₂, thus shifting the pH upwards to levels of pH preferably at least 6.6, more preferably at least 6.8, even more preferably 7 or higher, most preferably at least pH 7.5. The advantage of postponing the desired pH shift to the actual spray drying is that preceding production steps are not hindered by premature gelling and therewith associated blocking of the manufacturing lines. An additional advantage is that the retentate can be subjected to more stringent heat conditions to ensure microbial desctruction since it is ultimately dried at much lower pH.

Also, it has been found that the method or process according to preferred embodiments of the invention yields a WPC powder that at least exhibits a pH that is increased compared to conventional WPC80s, i.e. having a pH of at least 6.6, preferably at least 6.8, most preferably 7.0 or higher, preferably at least 7.5, having an increased gel strength that can conveniently be applied in all kinds of food applications, for instance in meat, confectionary, (fermented) dairy and bakery applications, for purposes of encapsulating ingredients and/or achieving satiety etc.

The production history of the (spray dried) WPC according to the invention is recognized by its (residual) carbonate content. In one aspect, the inventors provide an improved carbonate detection method using lactoferrin (LF), preferably bovine LF as an indicator. The method renders it possible to detect CO₂ levels and changes therein with improved resolution compared to CO₂ detection methods existing in the art. The method may be applied in all kinds of applications—also outside the field of foodstuffs—where CO₂ detection is desired. It is found particularly useful in the context of the invention to assess whether the preparation of the ultrafiltered and spray dried WPC involved carbonate salts route as described above. It is preferred that at least a predominant part of the pH increase, preferably all of the pH increase is achieved by using carbonate salt(s). Worded alternatively, it is particularly preferred that the carbonate salt(s) is/are the only base added to the acidified whey in the process to obtain the powder.

Food applications in which WPC powders and carbonates are combined in food applications are known, one of them being the baking of cake which involves the addition of soda for setting. However, it is noted that thus far in such processes, for instance as described in U.S. Pat. No. 4,421,777, the WPC is still that prepared by conventional alkali-based production methods. The later addition of bicarbonates together with the WPC powder does not change the functional properties of the WPC, these are fixed in the actual preparation of the powder itself. The inclusion of carbonate salts in manufacturing prior to (spray)drying is visibly distinct in the WPC thus formed compared to those cases where WPC powders are combined (“dry-mixed”) with carbonate salts. In the latter case, carbonate crystals are observed.

### DESCRIPTION OF THE INVENTION

In a first aspect, the invention thus pertains to a process for manufacturing WPC having improved functional properties from whey, preferably obtaining WPC having a (whey) protein content of at least 70 wt %, preferably 80-90 wt %, based on its dry weight, said process involving providing acidified whey, increasing the pH of said acidified whey using one or more carbonate salt(s), preceded and/or followed by ultrafiltration, and subjecting the ultrafiltered carbonate-containing whey to spray drying, to obtain a whey protein concentrate (powder). Worded differently, the invention pertains to a process of manufacturing WPC, wherein acidified whey is provided and subjected to ultrafiltration and spray drying, and wherein the pH of the whey is increased using carbonate salt before spray drying, i.e. before and/or after ultrafiltration. Hence, ultrafiltration may be carried out at the reduced pH or, alternatively, following the pH increase at a more neutral pH.

Following ultrafiltration but prior to spray drying, the retentate may optionally or mandatorily be subjected to diafiltration (DF) for further removal of lactose and minerals.

The retentate obtained after ultrafiltration and optional diafiltration preferably has a protein concentration of at least 60 wt %, more preferably at least 70, 80, 90, and most preferably at least 95%, based on dry solids weight. The total dry solids level of the retentate preferably ranges from 15-35 wt %, more preferably from 20-30 wt. % of the total weight of the retentate.

In the context of the invention, it is noted that the term “acidified whey” is not considered limited to “acid whey”, the term commonly applied in the field to distinguish the whey obtained in acid casein production from sweet whey (i.e. the normal by-product of cheese and rennet casein manufacture following the separation of the curds). Simplified, acid whey is obtained from acid coagulation of milk, while sweet whey is derived by rennet coagulation of protein (casein) from milk. Here, either sweet whey or acid whey can be used as a starting material, although acid whey is preferred. Acid whey has a pH of about 4-5, and sweet whey has a pH of about 5.8-6.8. Here, the term “acidified whey” is construed to mean whey that is acidified to a pH in the range of below 6.0, preferably 2.5-5.7, more preferably 2.5-5.0, most preferably 3.0-4.6. The pH reduction is achieved using one or more food grade acid(s), such as hydrochloric acid, sulphuric acid or citric acid.

In one embodiment, the acidified whey is provided by sweet whey being subjected to pH reduction. However, in a preferred embodiment, the source of whey is milk, where casein is removed by acid coagulation. Hence, in this embodiment, the acidified whey is acid whey. Contrary to sweet whey production, acid whey manufacture already involves a pH reduction step. However, starting from acid whey as obtained in acid casein production, a further pH adjustment may be required.

The pH increase after spray-drying preceded and/or followed by ultrafiltration preferably results in an increase of the acidified whey to a pH of at least 6.6, more preferably at least 6.8, even more preferably 7.0 or higher, preferably at least 7.4. It is preferred to increase the pH such that the end pH is lower than 8.5, preferably lower than 8.0. At higher pH levels whey protein degradation and Maillard reaction play a role. Also, high pH limits the shelf life and taste of the product.

It is considered within the skilled person's ambit to determine the pH adjustment required to achieve a certain pH shift upon spray drying. However, given the above preferred end pH ranges, the required pH prior to spray drying is preferably calculated from the linear relationship pH_{after spray drying}=2.21*pH_{before spray drying}−6.8. This relationship is determined for about 25% wt. WPC concentrations but can readily be determined for different conditions. It is more preferred that the pH prior to spray drying is adjusted using carbonates to at least pH 6.3. It is particularly preferred to add carbonate salts to a pH prior to spray drying of between 6.4 and 7.0. It is found that preferably between 70 and 80% of the originally added sodium carbonates is lost during spraying.

It is essential that the pH increase is established using one or more carbonate salts, preferably sodium, potassium, ammonium and/or calcium carbonates and/or bicarbonates. As explained above, during the evaporation step part of the carbonates present in the ultrafiltered whey is released in the form of CO₂, which in turn results in the desired pH increase. In one embodiment, at least 60, wt %, preferably at least 70 wt %, more preferably at least 80%, even more preferably at least 90%, particularly all base materials added is a carbonate salt. It is possible that the remainder is formed from alkali salts. In one embodiment, an initial pH increase is achieved using e.g. alkali salt, preferably sodium or potassium hydroxide, after which the carbonate salts are added to achieve the pH levels ultimately strived for. This way, any disadvantageous effects of initial CO₂ gas bubble formation can be reduced.

Ultrafiltration is in accordance with traditional methods known in the field, preferably carried out using a 1,000-50,000 D molecular weight cut-off (MWCO) membrane. In a particularly preferred embodiment of the invention the membrane will have a MWCO less than 10,000 D. Diafiltration may be applied after ultrafiltration to remove lactose and minerals.

If it is desired to obtain a low fat WPC or defatted WPC, the acidified whey may have been subjected to microfiltration. Preferably, the microfiltration is carried out using a micro filtration membrane with porosity in the range of 0.05-10 microns.

If not clear from the above, it is repeated that the invention does not rest in modifying ultrafiltration, microfiltration, diafiltration and spray-drying techniques as these are conventionally applied in the field to obtain existing WPCs. Modifications or changes therein without departing the scope of the invention are considered to fall within the ambit of the skilled person's knowledge.
Free (ionic) calcium may have a negative effect on the gelling properties. The functionality (gelling properties) of the WPC may, in an embodiment, thus be further improved by adding calcium binding agents like citrates or other di- or tri-valent organic carboxylic acids, phosphates, casein phosphopeptides (CPP), EDTA, and the like, to the acidified whey or retentate. Preferably the calcium binding agent is citrate and/or citric acid. Adding calcium binding agents may be done before or after ultrafiltration or diafiltration, in any event preferably before the spray drying step. Preferably, the calcium binding agent is added simultaneously with the carbonate salt; this way, pH variations are limited as opposed to adding carbonate salts and calcium binding agents at various stages in the process.

In one embodiment, the calcium binding agent(s) is(are) added before the carbonate. This offers the advantage of being able to add more carbonate to the retentate, giving a higher pH increase upon spray drying.

It is preferred that calcium binding agents are added in such an amount that the free calcium level in the dried WPC is below about 1000, preferably below about 800 ppm. In another embodiment, calcium binding agents may be added in an amount of 80-120% of the amount of total calcium present, on a molar/molar basis.
As mentioned earlier, in one embodiment, the upward pH adjustment may be performed before or after ultrafiltration or ultrafiltration and diafiltration In one embodiment, it may be preferred that the upward pH adjustment is carried out before ultrafiltration, or before ultrafiltration and diafiltration, thus yielding the additional advantage of a reduced mineral content, due to subsequent partial removal of the added sodium, potassium and/or calcium during ultrafiltration, or during ultrafiltration and diafiltration.

In another embodiment, the upward pH adjustment is performed after ultrafiltration or ultrafiltration and diafiltration. This has the advantage of executing a more efficient UF/DF process.

In yet another embodiment, a partial upward pH adjustment of the UF/DF retentate, preferably to 5.8-6.2, using alkaline agents, e.g. bases or basic salts, may be carried out before the calcium binding agent and carbonate are added. A subsequent pH adjustment is performed afterwards.

It will be appreciated that drying of the concentrate can be carried out by any suitable means, in addition to spray-drying. The temperature settings of the spray drier in the spray drying process are preferably adjusted in such a way that no thermal damage to the WPC is done; it is preferred maintain the inlet air temperature of the spray drier at less than 180° C.; more preferably, the inlet air temperature of the spray drier is 160° C. or lower, most preferably 150° C. or lower or even 140° C. or lower. The lower temperature limit may easily be assessed by the skilled person operating a spray drier. The outlet air temperature of the spray drier is preferably lower than 110° C., more preferably lower than 100° C., most preferred is lower than 90° C.

In another aspect, the invention pertains to a whey protein concentrate, preferably in the form of a powder, having improved functional properties. When dissolved in water, for instance at 25 wt %, the WPC according to the invention has a pH including and greater than 6.6, more preferably at least 6.8, most preferably at least 7.0, preferably greater than 7.4. For reasons outlined above a maximum pH of less than 8.5 or even less than 8.0 is preferred. The preferred carbonate content of the WPC ranges from 0.5 to 1.7 wt %, more preferably 0.7-1.4%—calculated in terms of the contribution of Na₂CO₃ equivalents to the total mass content of the WPC. It includes CO₂.

The WPC preferably has a (whey) protein content greater than 70% by weight, more especially of the order of 80-90 wt %, based on dry matter. It preferably shows gel strengths which are maintained or even increase upon increasing the salt levels, e.g. from 0 to 2% NaCl in the gel test solution. According to one aspect, there is provided a whey protein concentrate having a gel strength—in terms of gel strength—greater than 6000 grams, preferably more than 6500 grams. These results are obtained by measuring the maximum force in compression using a Texture Analyser [TA-XT2i, Stable Micro Systems] at compression speed=0.30 mm/s, distance 8.0 mm, T=25° C. More details are given in example 1b. For sake of comparison, these numbers are obtained for an aqueous composition containing 15% WPC solids and 2 wt % NaCl, which WPC has been maintained at 75° C. for 1 hour.

The powder is understood to comprise preferably less than 10%, more preferably less than 5% water. It is preferably a free flowing powder.

It is preferred that the WPC is obtained from acidified whey. The preferred calcium content corresponding therewith is preferably lower than 2500 ppm, preferably lower than 2300 ppm, more preferably lower than 2000 ppm, based on the WPC mass content. The calcium may have an effect on the gelling properties. The functionality (gelling properties) of the WPC may be further improved by including calcium binding agents like citrates, phosphates, CPP and the like. Suitable amounts (wt/%) include 0.2-2.0%, preferably 0.3-1.5%, more preferably 0.4-1.2%. As discussed before, this will reduce the level of free ionic calcium that disadvantageously affects the gelling behaviour of WPC.

In a preferred embodiment, the Na/Ca ratio (wt/wt) in the WPC powder may be at least 6.5, up to 14.0, as it has been found that within these ranges very high gel strength is obtained.

The invention also pertains to the use of the WPC according to embodiments of the invention in (the manufacture of) food applications, particularly in bakery, confectionary (fermented) dairy products, nutritional applications (satiety), functional food and encapsulation methods (encapsulation of e.g. fish oil, as an encapsulating agent). The high-gelling WPC of the invention finds particular application in fish and meat products, examples being cooked meats, hamburgers, patés and sausages, and Japanese fish products like surimi, kamaboko, chikuwa, hanpen. Also, the WPC according to the invention can suitably be used as egg white replacer. In one aspect, the invention pertains to a method for treating satiety, by administering the WPC according to the invention.
In another aspect, the invention pertains to a carbonate detection method making use of the link between iron stabilisation of lactoferrin and absorption. Although the inventors do not wish to be bound by any theory, they believe that the success of the method relates to the fact that carbonates facilitates iron binding at the LF binding site. Since the binding of iron to LF is accompanied from a proportional increase of absorption at and around 465 nm, the carbonate levels can be calculated according to a calibration curve. Both the calibration and the actual measurement are performed at buffering conditions, preferably using potassium phosphate buffer, to exclude effects from other proteins. In one embodiment, the invention pertains to a method for determining the amount of carbonate in a composition, by (i) bringing a sample of the composition into contact with a pre-determined amount of iron-stabilized lactoferrin under aqueous conditions, determining the absorption at a fixed wavelength once the absorption level at said wavelength stabilizes, preferably within 10 minutes, (ii) comparing said absorption level with a database/calibration curve, and (iii) calculating the amount of carbonate salts there from. The method is outlined in example 4. A suitable wavelength is found in the range of 450-480 nm, more preferably at 455-475 nm.

### EXAMPLES

### Example 1a

### Preparation WPC without Addition of Calcium Binder-Citric Acid

Acid whey, obtained from caseinate production, was subjected to ultrafiltration and diafiltration. Ultrafiltration was carried out with 10 kDa PES membranes (HFK-131, Koch, USA). Ultrafiltration was carried out to a concentration factor of 20-25 and with a diafiltration degree of about 30%, resulting in an acid whey retentate product with a dry solids content of about 27%, a protein/total solids of about 80%, a pH of 4.4-4.6, and a temperature of about 8° C. Then, about 100 mmol Na₂CO₃ was added per 1 kg WPC of 26.7% dry solids [3.97% Na₂CO₃ based on dry weight], and the pH was thus adjusted to pH 6.51. The aqueous composition was then subjected to spray-drying (inlet air temperature 145° C., outlet air temperature 99° C.), to obtained a powdered form. The end pH after spray drying (for 25% WPC-solution) was pH 7.72, corresponding to 1.0% Na₂CO₃ remaining. It contained minimum amounts of water, about 5%. The protein content was above 75%, (about 80%) and the amount of ash was about 4.9% as measured at 550° C., according to NEN 6810.

### Example 1b

### Preparation WPC with Addition of Calcium Binder-Citric Acid

Example 1a was repeated, with exception that, after ultrafiltration, about 100 mmol citric acid 1.0 M aqueous solution was added. Subsequently, 94.5 mmol Na₂CO₃ was added per 1 kg WPC of 26.7% dry solids, and the pH was thus adjusted to pH 6.35. Spray-drying conditions were identical to those of example 1a. The end pH after spray drying (for 25% WPC-solution) was pH 7.30. The powder contained minimum amounts of water, about 5%. The protein content was above 75%, (about 80%) and the amount of ash was about 5.71% as measured at 550° C., according to NEN 6810.

### Example 1c

### Gelling Properties

A 15% w/w WPC aqueous solution (98 ml) was prepared in a 137 ml plastic cup (cup dimensions: 5 cm diameter, height 7 cm; height of the liquid was 5 cm) either in presence or absence of NaCl, and with or without pH adjustments. The solutions were subjected to heating for 1 hour at 75° C., and subsequently cooled and stored at 4° C. overnight.

Prior to measurement with a Texture Analyser [TA-XT2i, Stable Micro Systems] samples were allowed to warm up to room temperature. A compression test was performed where the gel strength (in grams) was determined. The probe dimensions were 45 by 40 mm, probe height was 160 mm. In this test the maximum force in compression was measured (compression speed=0.30 mm/s, distance 8.0 mm, T=25° C.). Results—in terms of the gel strength—for the WPCs of example 1a and 1b are shown in Table 1, and compared to results obtained starting from WPC80, as it is commercially available by the name “Textrion™ PROGEL 800” with DMV International. The pH adjustments were performed using 1 M NaOH.

TABLE 1
gelling properties

Gel strength [g]

Textrion ™ PROGEL 800

| pH as is (pH 6.6) + | 7297 | 6143* |
|---|---|---|
| 2% NaCl | 2882 | 1088 |
| | WPC (ex 1a) | WPC (ex 1b) |
| pH as is (pH 7.7) + | 7049 | 9800 |
| 2% NaCl | 8963 | 8600 |

*The results in columns II and III are obtained for different batches of Textrion ™ PROGEL 800. The same trends are observed.

It is concluded that the use of WPC according to embodiments of the present invention results in an increased gel strength upon salt addition, whereas a dramatic drop in gel strength was observed for a non-modified WPC. The properties at increased salt strengths are particularly important in meat applications, where the salt simulates meat conditions.

### Example 2

### Applications

The WPC obtained in example 1a was tested in an eggless sponge cake recipe, and compared with sponge cake obtained with WPC 80 (Textrion PROGEL 800®, commercially available with FrieslandCampina DMV; abbreviated as TP800), and with egg-based sponge cake (traditional recipe). For sake of convenience, the recipes are included in table 2.

The sponge cake thus obtained was then analyzed for textural properties, the results are summarized in tables 3a and 3b. The sponge cake obtained using WPC according to example 1 showed better performance than normal WPC80, closer resemblance to the traditional sponge cake. This is attributed to it better gelling properties.

### Example 3

### Preparation Method for Sausages

Pork sausages were prepared according to the following recipe:

| Ingredients | Reference | WPC addition |
|---|---|---|
| Minced pork | 100 | 100 |
| Cold water (0° C.) | 56 | 46 |
| NaCl | 2.4 | 2.4 |
| Meat-curing agent* | 1.6 | 1.6 |
| WPC ex 1b or Textrion Progel 800 | | 10.0 |

*sodium polyphosphate 20.0%, sodium pyrophosphate (anhydride) 20.0%, sodium acid pyrophosphate 10.0%, 1-ascorbic acid 5.0%, sodium nitrite 1.2%

The pork meat was comminuted using a meat mincer fitted with a 5 mm cutting plate (in the art called a “Wolf,” e.g. a K+G Wetter Wolf, obtainable from Möller & Co., The Netherlands) and divided in 5 kg portions. The remaining ingredients were dissolved in the water. The pork and water were stirred in a Hobart mixer (speed 1) for two minutes, then 10 seconds on speed 2. The pork dough was then filled in conventional sausage casings (diameter 4 cm). Next, the casing were warmed in hot water of 75° C. for 1 hour. The casings were then cooled, and after 24 hours resting time, sliced in 5 cm pieces and the slices were placed in plastic cups (as in example 1).

The gel strength of the slices was measured using the method in example 1, and the results plotted in Table 4. The results show that the gelling properties improved significantly (20%) compared to those obtained for conventional WPC.

TABLE 2
sponge cake recipe

| | Cake description | | | | | |
|---|---|---|---|---|---|---|
| | Standard | | Standard | | Standard | |
| | eggless recipe | | recipe with | | sponge recipe | |
| | with TP800 | | WPC (ex 1) | | with egg | |
| | % | g | % | g | % | g |
| Flour | 15.9 | 111.2 | 15.9 | 111.2 | 18.0 | 160.0 |
| Sugar | 22.3 | 155.8 | 22.3 | 155.8 | 25.8 | 230.0 |
| Starch | 11.1 | 77.9 | 11.1 | 77.9 | 7.0 | 62.0 |
| Baking powder | 0.7 | 4.6 | 0.7 | 4.6 | 0.4 | 4.0 |
| BV 46* | 4.0 | 27.8 | 4.0 | 27.8 | 3.4 | 30.0 |
| Water | 39.7 | 278.2 | 39.7 | 278.2 | 14.6 | 130.0 |
| Textrion PROGEL | 6.4 | 44.5 | 6.4 | 44.5 | 30.9 | 275.0 |
| 800 ®/WPC (ex. 1a)/egg | | | | | | |

*BV 46 is a batter stabilizer obtainable from FrieslandCampina Kievit.

TABLE 3a
Properties sponge cake

| | | | Standard sponge |
|---|---|---|---|
| Cake description | TP800 | WPC (ex 1) | recipe |
| Reaction time (min:sec)* | 4:15 | 3:45 | 3 |
| Overrun (%) | 256 | 245 | 240 |
| Penetration (mm) | 6.1 | 6.6 | 11 |
| Remarks: | Crème | slightly yellowish | Yellow |

*Reaction time: time needed until max. overrun is reached

TABLE 3b
Properties sponge cake

| | | | Standard sponge |
|---|---|---|---|
| Cake description | TP800 | WPC (ex 1) | recipe |
| Volume (L) | 2.26 | 2.7 | 3.1 |
| Texture Analyzer: | 459.1 | 411.8 | 254.4 |
| Firmness, Force 1 (grams) | | | |
| Texture Analyzer: | 445.5 | 404.2 | 238.4 |
| Firmness, Force 2 (grams) | | | |
| Texture Analyzer: | 95.4 | 93.85 | 96.0 |
| Elasticity | | | |
| Height (mm) | 47 | 53 | 59 |
| Cake Weight (g) | 458 | 461 | 504 |
| Cake density | 202.7 | 171.4 | 165.3 |
| (weight/volume) | | | |

TABLE 4
Gel strengths.

| Sample | Gel strength (g)* |
|---|---|
| Reference (no WPC) | 1618 |
| WPC ex. 1b | 4286 |
| Textrion 800 | 3575 |

*It is noted that the gel strength is based on 6.2% WPC solids, and may not be compared to the gel strength measured for 15% WPC solids.

### Example 4

### Carbonate Detection Method

A commercially available lactoferrin (LF) powder (obtained from DMV-international, Veghel) was dissolved in 50 mM potassium phosphate, 150 mM NaCl, pH9.5) to achieve a concentration of 4% (w/w). The pH was adjusted to 9.5 with 2N NaOH.

Separately, a WPC sample was dissolved in 50 mM potassium phosphate buffer, 150 mM NaCl, pH 9.5 to 25% (w/w). Subsequently, the pH of the solution was adjusted to 7.0-7.2 with 1 N HCl solution and the solution was heat-treated at 90° C. for 30 minutes in a sealed bottle. The obtained gel was milled and dispersed in equal amount of 50 mM potassium phosphate buffer, 150 mM NaCl, pH 9.5 (as the gel) and the mixture was heat-treated in a sealed bottle at 90° C. for 30 minutes. The subsequent centrifugation at 10000 rpm (˜5600 g) for 15 minutes, resulting in the separation of the gel from the supernatant.

The supernatant solution was then added to the LF-containing solution to reach 2% LF, 35 mM phosphate buffer, 150 mM NaCl and pH 9.5. By comparing the profile of iron binding with a pre-determined standard profile, the carbonate concentration could be readily calculated. Thereto, 3.4 ml samples were transferred to a 4 ml 1 cm plastic curvette containing 100 μl 33.3 mM FeCl₃, and the extinction was followed at 465 nm at t=0 and after 3 minutes. The increase of absorption at 465 nm during these 3 minutes was expressed as ΔA465 nm=A465 nm (t=3)-A465 nm (t=0).

Independently, a calibration curve was taken form a series of points: Different amounts of 1 M Na₂CO₃ (0, 15, 30, 45, 60, 75, 90 μl) were added to 30 gram 2% LF solution, 50 mM potassium phosphate buffer, 150 mM NaCl, pH9.5. After mixing, the pH was adjusted to 9.5 with 1 N HCl. Absorption profiles were taken for these samples likewise. The absorption level obtained for the WPC-sample could be recalculated to the corresponding Na₂CO₃ concentrations from this curve.

The method was tested for its accuracy by adding different amounts of sodium carbonate to WPC prior to spray drying, and measuring the pH before and after spray-drying. The second entry corresponds with example 1. To exclude any effect of the WPC, the measurements were repeated using different WPCs. From the pH, the amount of carbonates was theoretically calculated and compared to the actual levels measured according to the above detection method. The results are summarized in table 5.

TABLE 5
Relationship pH and Na₂CO₃ level

| | Added Na₂CO₃ | Added | | End pH | | | |
|---|---|---|---|---|---|---|---|
| Added | (mmol) for 1 kg | Na₂CO₃ | Start pH | after | Remaining | | |
| Na₂CO₃ | WPC 26.7% | (%, on | before | spray- | Na₂CO₃ | Na₂CO₃ | |
| (mmol) | (dry solids) | dry solid) | drying | drying | calculated | Left | Measured |
| 18 | 90 | 3.57% | 6.38 | 7.29 | 0.89% | 24.9% | 0.89% |
| 20 | 100 | 3.97% | 6.51 | 7.72 | 1.06% | 26.7% | 0.95% |

Table 5 shows that the amount of carbonate can be determined even if present in very small amounts, with acceptable standard deviations.

1-14. (canceled)

15. A process for manufacturing whey protein concentrate (WPC) from whey, the process comprising:
(a) increasing the pH of acidified whey using one or more carbonate salt(s),
(b) subjecting the acidified whey to ultrafiltration before and/or after increasing the pH, and
(c) subjecting the ultrafiltered whey to spray drying.

16. The process according to claim 15, wherein the pH is increased to at least 6.3.

17. The process according to claim 16, wherein the pH is increased to between 6.4-7.0.

18. The process according to claim 15, wherein the pH of the whey after spray drying is higher than 7.0

19. The process according to claim 18, wherein the pH of the whey after spray drying is between 7.0 and 8.5.

20. The process according to claim 15, wherein the retentate obtained after ultrafiltration is subjected to diafiltration.

21. A whey protein concentrate (WPC) having (i) a whey protein content of at least 70%, based on dry matter, (ii) a pH of at least 6.6, and (iii) a carbonate content of 0.7-1.4%, calculated in terms of the contribution of Na₂CO₃ equivalents to the total weight.

22. The WPC according to claim 21, having a pH of at least 7.0.

23. The WPC according to claim 21 in the form of a powder.

24. The WPC according to claim 21, having a pH between 7.0 and 8.5.

25. The WPC according to claim 21, in the form of a gel, wherein the gel has a gel strength of at least 6000 grams, by measuring the maximum force in compression using a Texture Analyser at compression speed=0.30 mm/s, distance 8.0 mm, T=25° C., for an aqueous composition comprising 15% WPC solids and 2 wt % NaCl, after 1 hour at 75° C.

26. The WPC according to any claim 21, having a calcium content of less than 2500 ppm, based on dry weight.

27. A food product comprising WPC according to claim 21.

28. The food product according to claim 27, wherein said food product comprises bakery, confectionary, fermented dairy, fish and/or meat.

29. The food product according to claim 28, wherein the meat is cooked meat, hamburger, paté, or sausage.

30. The food product according to claim 28, wherein the fish is surimi, kamaboko, chikuwa, hanpen.

31. A method of preparing a food product comprising applying WPC according to claim 27 to bakery, confectionary, fermented dairy, fish and/or meat.

32. A method for determining the amount of carbonate salts in a composition, the method comprising:
(i) contacting a sample of the composition with a pre-determined amount of iron-stabilized lactoferrin under aqueous conditions,
(ii) determining the absorption of the sample at a fixed wavelength,
(iii) comparing the absorption level with a database/calibration curve, and
(iv) calculating the amount of carbonate salts in the composition.
